(19) United States
(12) Patent Application Publication
(10) Pub. No.: US 2009/0291974 A1
(43) Pub. Date: Nov. 26, 2009

(54) BOSENTAN SALTS

(76) Inventor: Jie ZHU, Nijmegen (NL)

Correspondence Address: SYNTHON IP INC, 7130 HERITAGE VILLAGE PLAZA, STE 202, GAINESVILLE, VA, 20155, US

(21) Appl. No.: 12/471,676

(22) Filed: May 26, 2009

Related U.S. Application Data
(60) Provisional application No. 61/055,699, filed on May 23, 2008.

(51) Int. Cl. A61K 31/506; C07D 403/04; A61P 9/12

(52) U.S. Cl. 514/269; 544/296

(57) ABSTRACT

Stable acid addition salts of bosentan useful for the purification of bosentan base; the salts are in solid state and the starting acid has a pKa lower than 3.

This application claims the benefit of priority under 35 U.S.C. § 119(e) from prior U.S. Provisional Application No. 61/055,699, filed May 23, 2008, the entire contents of which are incorporated herein by reference.

### BACKGROUND OF THE INVENTION

1. Field of the Invention

The present invention relates to acid addition salts of bosentan, pharmaceutical compositions containing the salts, and methods of purifying bosentan base using the salts.

2. Description of the Prior Art

Bosentan is a pharmaceutically active compound (an endothelin receptor antagonist) useful for the treatment of pulmonary arterial hypertension and is represented by the formula (I).

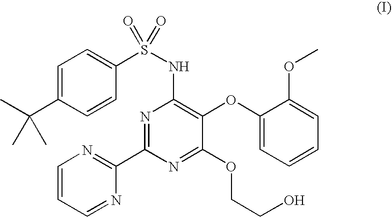

The marketed pharmaceutical composition is an immediate release tablet sold under the brand name TRACLEER® (Actelion Pharmaceuticals US, Inc., South San Francisco, Calif.), that contains bosentan as a monohydrate of the free base (bosentan monohydrate). Bosentan was disclosed in EP 526,708 (U.S. Pat. No. 5,292,740) and was generically described as including salts thereof, but only the sodium salt of bosentan was shown

Bosentan monohydrate is freely soluble in acetone and dichloromethane, soluble in ethanol and ethyl acetate, slightly soluble in methanol and isopropanol, and very slightly soluble in hexane. Bosentan is poorly soluble in water (1.0 mg/100 ml) and in aqueous solutions at low pH (0.1 mg/100 ml at pH 1.1 and 4.0; 0.2 mg/100 ml at pH 5.0). The solubility of bosentan increases at higher pH values (43 mg/100 ml at pH 7.5).

U.S. Pat. No. 5,292,740 discloses a process of preparing bosentan using a chloro-intermediate of formula (II).

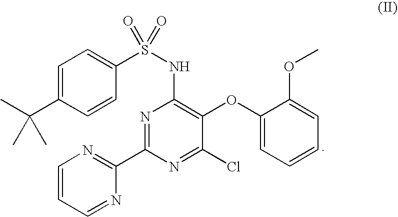

This process is apparently accompanied by the formation of impurities that are difficult to remove. U.S. Pat. No. 6,136,971 discloses another process of preparing bosentan from the compound of formula (II), which attempts to avoid the impurity problem by using protecting/deprotecting steps.

It would be desirable to have additional purification techniques for preparing bosentan of a high purity, particularly bosentan monohydrate of a pharmaceutical grade.

### SUMMARY OF THE INVENTION

The present invention relates to the discovery of stable acid addition salts of bosentan that are useful for the purification of bosentan base as well as in pharmaceutical compositions. Accordingly, a first aspect of the invention relates to an acid addition salt of bosentan, wherein said salt is in solid state and wherein said acid has a pKa lower than 3. The acid can be preferably selected from hydrochloric acid, hydrobromic acid, methane sulfonic acid, benzene sulfonic acid, sulfuric acid, p-toluenesulfonic acid, oxalic acid, and maleic acid. The solid state includes crystalline as well as amorphous states. Typically the solid state bosentan acid addition salt is a monovalent salt having an acid:base ratio of about 1:1. Advantageously, the bosentan acid additional salt is selected from bosentan monohydrochloride and bosentan p-toluenesulfonate, preferably from crystalline bosentan monohydrochloride and crystalline bosentan p-toluenesulfonate.

Another aspect of the present invention relates to a pharmaceutical composition comprising the solid state bosentan acid addition salt described above and at least one pharmaceutically acceptable excipient.

Yet another aspect of the present invention relates to a method of making an acid addition salt of bosentan, which comprises combining bosentan base and an acid having a pKa lower than 3 in an organic solvent, preferably a polar organic solvent, to form a solution; precipitating a bosentan acid addition salt from said solution; and optionally isolating the precipitated bosentan acid addition salt.

A further aspect of the present invention relates to a method of purifying bosentan, which comprises combining crude bosentan and an acid having a pK of about 3 or less in a first solvent to obtain an acid addition salt of bosentan; isolating said acid addition salt of bosentan from said first solvent; converting said bosentan acid addition salt into bosentan base in a second solvent; and isolating said bosentan base from said second solvent.

Another aspect of the invention relates to a process of purifying bosentan salt that comprises dissolving a solid bosentan acid addition salt in an organic solvent, and precipitating said salt to obtain a purified solid bosentan acid addition salt.

### BRIEF DESCRIPTION OF THE DRAWINGS

FIG. 1 is an XRPD pattern for the amorphous bosentan HCl salt of Example 3;

FIG. 2 is a DSC curve for the amorphous bosentan HCl salt of Example 3;

FIG. 3 is an XRPD pattern for the crystalline bosentan HCl salt of Example 4; and

FIG. 4 is a DSC curve for the crystalline bosentan HCl salt of Example 4.

### DETAILED DESCRIPTION OF THE INVENTION

Bosentan's structure is quite complex, having both acidic and basic centers (having an overall pKa of 5.46). While a salt based on an acidic center is known (i.e., the sodium salt of bosentan), the present invention relates to the discovery that stable acid addition salts of bosentan can be made. These acid addition salts are useful for the purification of bosentan base and also directly useful in pharmaceutical compositions. The bosentan acid addition salts of the invention are made from fairly strong acids having a pKa of about 3 or less, typically about 2 or less. The “pKa” refers to the pKa of the starting acid; hence as used herein reference to the pKa even in the context of the addition salt is referring to the pKa of the starting acid. Suitable acids include, for example, hydrochloric acid, hydrobromic acid, methane sulfonic acid, benzene sulfonic acid, sulfuric acid, p-toluenesulfonic acid, oxalic acid, and maleic acid.

The bosentan acid addition salts of the invention are isolatable in a solid state, which can be advantageous. The “solid state” includes crystalline and amorphous forms, as well as mixtures thereof, and also includes solvates and hydrates. Generally the acid addition bosentan salts of the invention can be obtained in a stable solid state form making them useful for purification, bulk storage, or use in pharmaceutical compositions and methods of treatment.

The bosentan acid addition salts of the present invention are typically monovalent salts, i.e., having an acid:base ratio of about 1:1. Analytical methods, such as titration or ionic chromatography, may show a ratio of acid:base of 0.8:1 to 1:1.2 in the isolated solid form of the salt as a result of, e.g., traces of unbound acid and/or base and inherent variance associated with the analytical method. Such variation in the acid:base ratio is encompassed by an acid:base ratio of “about 1:1.”

Exemplary bosentan acid addition salts according to the present invention include bosentan hydrogensulphate, bosentan oxalate, bosentan maleate, bosentan hydrochloride, bosentan methane sulfonate, bosentan benzene sulfonate, and bosentan p-toluenesulfonate. In a preferred embodiment, the bosentan acid addition salts of the present invention are bosentan monohydrochloride and bosentan p-toluenesulfonate. Each of these salts is isolatable in a crystalline solid state with a molar ratio of bosentan to acid moieties of about 1:1.

The bosentan acid addition salts of the present invention can be made by combining bosentan base and an acid having a pKa of 3 or less in an organic solvent, preferably a polar organic solvent, to form a solution, and then precipitating a bosentan acid addition salt from said solution. Optionally the precipitated bosentan acid addition salt can be isolated. The “isolated form” means a product which is substantially free from solvents and reagents used in the process of making it, not including any solvent and/or reagent that are firmly bound in a definite amount within the crystalline lattice of the solid material to form specific physical forms such as hydrates, solvates and/or clathrates.

A molar equivalent or a slight excess of the starting acid with reference to the bosentan base is typically used in order to form a bosentan acid addition salt having an acid:base ratio of about 1:1.

The bosentan base used in forming the bosentan acid addition salt (i.e., the starting bosentan base) can be any form of bosentan base, including bosentan hydrate (bosentan monohydrate), in any degree of purity. The starting bosentan base can also be crude bosentan that is present in the reaction mixtures obtained after the chemical synthesis of bosentan.

The organic solvent used is typically a polar organic solvent, which includes both protic and aprotic solvents. Generally, the dielectric constant of a solvent provides a rough measure of a solvent's polarity; solvents with a dielectric constant of less than 15 are typically considered nonpolar. Examples of suitable polar solvents include C3-C10 aliphatic ketones (e.g., acetone, methyl tert.butyl ketone, etc.), C1-C6 chlorinated hydrocarbons (e.g., dichloromethane), C1-C6 aliphatic alcohols (e.g., methanol, ethanol, isopropanol), C3-C10 aliphatic esters (e.g., ethyl acetate), C2-C5 aliphatic nitriles (e.g., acetonitrile), and ethers including cyclic ethers (e.g., di-isopropyl ether, tetrahydrofuran), as well as mixtures thereof.

There is no specific order in which the bosentan base and the acid must be combined in the solvent to form the solution. Generally the conditions are such that all of the bosentan (and all of the acid) is dissolved in the solvent, though strictly speaking such is not required; i.e., some amount of solid or immiscible bosentan may be present in the solution. The dissolution of bosentan base in the solvent is advantageously performed at an enhanced temperature, which includes the reflux temperature of the solvent. The contacting or combining of the bosentan-containing solvent with the acid is advantageously performed at an ambient or higher than ambient temperature, including the reflux temperature of the solvent. In other embodiments, the acid can be added, e.g., substantially at the same time as the base, before the base, etc.

The precipitation of the bosentan acid addition salt can be carried out in various ways. For example, the precipitation can occur spontaneously upon the contacting of the bosentan with the acid in the organic solvent. Precipitating of the bosentan acid addition salt can also be induced by seeding the solution, cooling the solution, evaporating at least part of the solvent, adding an antisolvent, and by combining one or more of these techniques.

The precipitated bosentan acid addition salt can be isolated from the solution by conventional techniques, e.g. filtering or centrifugation, and can be washed and dried.

The isolated bosentan acid addition salt can, however, be purified if desired. For example, the isolated salt is recrystallized or reprecipitated by dissolving (at least partially, e.g., suspending) the isolated salt in a solvent, such as any of the above defined polar organic solvents, at an enhanced temperature (which includes a reflux temperature of the solvent), and then crystallizing or precipitating the salt from the solvent. The recrystallization (reprecipitation) process may be repeated until a desired purity of the isolated bosentan acid addition salt is obtained. For clarity, the terms “purify,” “purification,” “purified,” and variations thereof are used herein to indicate an improvement in the quality or purity of the substance and are not meant in the narrow sense of obtaining near absolute purity. Hence reducing the impurities from 2.0% to 1.5% represents a “purification” of the substance.

The solid state bosentan acid addition salts of the present invention can be advantageously used to obtain purified bosentan. In general, crude bosentan can be purified by converting it to a bosentan acid addition salt as described above and then converting the bosentan salt back into bosentan base. For example, a purification process can comprise (i) combining crude bosentan and an acid having a pKa of about 3 or less in a first solvent, preferably a polar organic solvent, to obtain an acid addition salt of bosentan; (ii) isolating the acid addition salt of bosentan in solid state from the first solvent; (iii) converting the bosentan acid addition salt into bosentan base in a second solvent, preferably an aqueous solvent; and (iv) isolating the bosentan base from said second solvent. Because structurally related impurities present in the crude bosentan are generally soluble in the organic solvents used to form the salt, these impurities generally remain in the first solution during the isolation of the solid bosentan acid addition salt; thereby separating these impurities from the bosentan moiety. The conversion to bosentan base, especially in an aqueous-based solvent, can likewise provide a further purification effect with respect to water-soluble impurities. “Crude bosentan” means a bosentan base or salt having insufficient purity and includes reaction mixtures obtained after the chemical synthesis of bosentan as well as bosentan having near pharmaceutical grade purity. From a practical standpoint, the crude bosentan is typically a bosentan base including hydrates and solvates thereof. Likewise, the produced “bosentan base,” which has an enhanced purity or quality relative to the crude bosentan, includes hydrates and solvates of bosentan base and specifically includes bosentan monohydrate. The above-recited process steps are not exhaustive; additional steps can also be included. For example, the acid addition salt of bosentan can itself be purified, such as by (re)crystallization as described above, before being converted to bosentan base.

The first solvent is generally a polar organic solvent as described above in the context of making the bosentan acid addition salts. Thus, examples of suitable first solvents include C3-C10 aliphatic ketones (e.g., acetone, methyl tert.butyl ketone, etc.), C1-C6 chlorinated hydrocarbons (e.g., dichloromethane), C1-C6 aliphatic alcohols (e.g., methanol, ethanol, isopropanol), C3-C10 aliphatic esters (e.g., ethyl acetate), C2-C5 aliphatic nitrites (e.g., acetonitrile), and ethers including cyclic ethers (e.g., di-isopropyl ether, tetrahydrofuran), as well as mixtures thereof.

The bosentan acid addition salt, which can be formed before or during precipitation thereof, is conveniently isolated as a solid from the first solvent by known techniques such as filtration, etc. The precipitation of the solid state acid addition salt of bosentan can be carried out by the techniques as described above.

The isolated solid bosentan acid addition salt can be converted into bosentan base by any suitable or convenient technique. Generally, the solid salt is dissolved and/or suspended in the second solvent and converted to base, optionally via the use of a base. The second solvent is preferably more polar than the first solvent. Advantageously the second solvent is an aqueous-based solvent in which bosentan base is insoluble. Such solvents include water as well as water-miscible solvents and combinations thereof. The base used to convert the salt of bosentan to bosentan base may be an organic or inorganic base and is preferably a base that binds the acid present in the second solvent to form a salt that is soluble in the second solvent. Suitable bases include sodium and potassium hydroxide. Upon addition of the base to the salt-containing second solvent, bosentan generally precipitates in a solid form, preferably as bosentan hydrate (bosentan monohydrate) when sufficient water is present. The precipitated and purified bosentan can then be isolated from the reaction mixture, e.g., by filtration or centrifugation, and is optionally washed and dried.

Conversion of the bosentan salt to the bosentan base does not require the use of a base, however. If the second solvent is water or comprises water, the bosentan salts of the present invention can be hydrolyzed by the water to form the bosentan base. Such reaction is normally spontaneous and the bosentan base generally readily precipitates from the aqueous solvent because of its low solubility in water. Of course the conversion can use both techniques; water for hydrolysis and the presence of a base.

In a preferred embodiment, the above purification process results in bosentan base having less than 1% impurities, more preferably less than 0.5%; e.g., at least 99.6% pure.

The acid addition salts of bosentan can also be formulated in pharmaceutical compositions. For instance, a suitable pharmaceutical composition may comprise a bosentan acid addition salt and at least one pharmaceutically acceptable excipient.

Pharmaceutically acceptable excipients are known in the art and include carriers, diluents, fillers, binders, lubricants, disintegrants, glidants, colorants, pigments, taste masking agents, sweeteners, flavorants, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, sugars, etc. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; polyalkylene glycols such as polyethylene glycol and polypropylene glycol; and polyacrylic acids including their copolymers and crosslinked polymers thereof, e.g., Carbopol® (B.F. Goodrich), Eudragit® (Rohm), polycarbophil, and chitosan polymers. Waxes include white beeswax, microcrystalline wax, carnauba wax, hydrogenated castor oil, glyceryl behenate, glycerylpalmito stearate, and saturated polyglycolyzed glycerate. Calcium phosphates include dibasic calcium phosphate, anhydrous dibasic calcium phosphate, and tribasic calcium phosphate. Sugars include simple sugars, such as lactose, maltose, mannitol, fructose, sorbitol, saccharose, xylitol, isomaltose, and glucose, as well as complex sugars (polysaccharides), such as maltodextrin, amylodextrin, starches, and modified starches.

The bosentan acid addition salts of the present invention are useful in treating cardiovascular disorders including hypertension, ischemia, vasospasms, and angina pectoris, by administering an effective amount thereof to a patient in need of such treatment. In particular the salts are useful in treating pulmonary arterial hypertension. Typically effective amounts range from 25 mg to 300 mg, expressed as the amount of base, per day in one or two portions.

The invention will be further described with reference to the following non-limiting examples.

### Example 1

### Preparation of Crude Bosentan and its HCl Salt

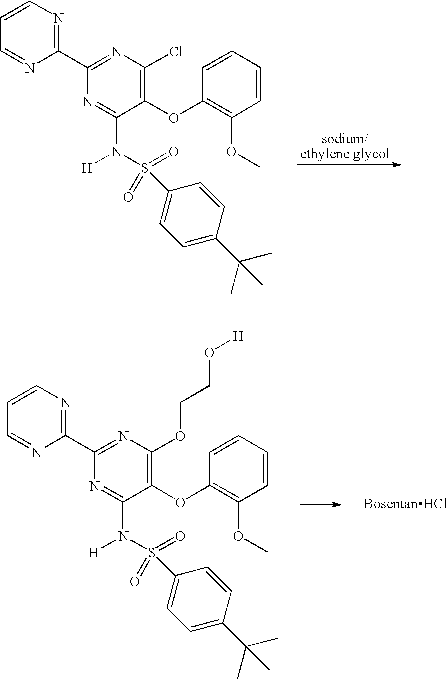

16.9 g of p-t-butyl-N-[6-chloro-5-(o-methoxyphenoxy)-4-pyrimidinyl]benzene-sulfonamide were added to a sodium glycolate solution made from 55 ml of ethylene glycol and 2.0 g of sodium. The reaction mixture was stirred at 95° C. for 2 hours. While cooling down to room temperature, 250 ml of H₂O and 400 ml of CH₂Cl₂ were added. The pH in the aqueous phase was adjusted to about 6. The mixture was further stirred at room temperature for 30 min. Layers were separated. The CH₂Cl₂ layer was stirred again with 250 ml H₂O for 30 min. The separated CH₂Cl₂ layer was washed again with H₂O (50 ml), dried over Na₂SO₄ and concentrated in vacuo to give crude bosentan as an oily material. Purity was about 97.0% based on HPLC.

The crude oily product was dissolved in 30 ml of 2-propanol at 40° C. While stirring, 6.5 ml of an HCl solution (about 5 N to about 6 N in 2-propanol) was added dropwise, and completed in about 5 min. The mixture was stirred for 2 hours; heating was stopped and bosentan HCl was seeded. 10 ml of 2-propyl ether was added, and the mixture was further stirred at room temperature for 2 hours.

A solid was filtered off and washed with 10 ml of 2-propyl ether/2-propanol (3/1). 15.1 g of a solid was obtained after drying at 40° C. in vacuo over night. Purity was 97.99% and max. Individual impurity was 1.08%.

M.p. about 145° C. to about 147° C.

### Example 2

### Purification of Bosentan HCl Salt

a. 15.1 g of bosentan HCl (purity of about 98.0%) was suspended in 30 ml of 2-propanol. The suspension was refluxed, while stirring, for 1 hour. Then the suspension was further stirred for 2 hours at room temperature. A solid was collected by filtration. 12.5 g of a solid was obtained after drying at 40° C. in vacuo over night. Purity was 99.04% and max. individual impurity was 0.55%.

M.p. about 144° C. to about 147° C.

b. A mixture containing 2.50 g of bosentan HCl (purity of about 96.55%) in 15 ml acetonitrile was warmed up to 50° C. to get a clear solution. The mixture was stirred overnight while cooling down to room temperature. A solid was filtered off and washed with acetonitrile (2 ml). 1.34 g of a solid was obtained after drying in open air overnight. Purity was 99.75% and max. individual impurity was 0.13%.

### Example 3

### Preparation of Amorphous Bosentan HCl Salt

17.0 g of p-t-butyl-N-[6-chloro-5-(o-methoxyphenoxy)-4-pyrimidinyl]benzene-sulfonamide were added to a sodium glycolate solution from 55 ml of ethylene glycol and 2.0 g of sodium. The reaction mixture was stirred at 95° C. for 1.5 hours. While cooling down to room temperature, 250 ml of H₂O and 400 ml of CH₂Cl₂ were added. The pH in the aqueous phase was adjusted to about 6. The mixture was further stirred at room temperature for 30 min. Layers were separated. The CH₂Cl₂ layer was stirred again with 200 ml H₂O for 30 min. The separated CH₂Cl₂ layer was filtered off via a celite layer, dried over Na₂SO₄, and concentrated in vacuo to give crude bosentan as an oily material (about 19.5 g). Purity was about 94% based on HPLC.

Crude material was dissolved in 100 ml of acetonitrile, while stirring at room temperature, and 10 ml of HCl (about 5 N to about 6 N in 2-propanol) was added. The mixture was seeded and stirred at room temperature for 2 hours. 50 ml of 2-propylether was added, and the mixture was further stirred for 2 hours. A solid was collected by filtration and washing with acetonitrile (15 ml). 9.56 g of a yellow solid was obtained after drying, (99.48% purity and max. individual impurity 0.15% based on HPLC). M.p. about 92° C. to about 110° C. XRPD indicated that the yellow solid was mainly an amorphous material.

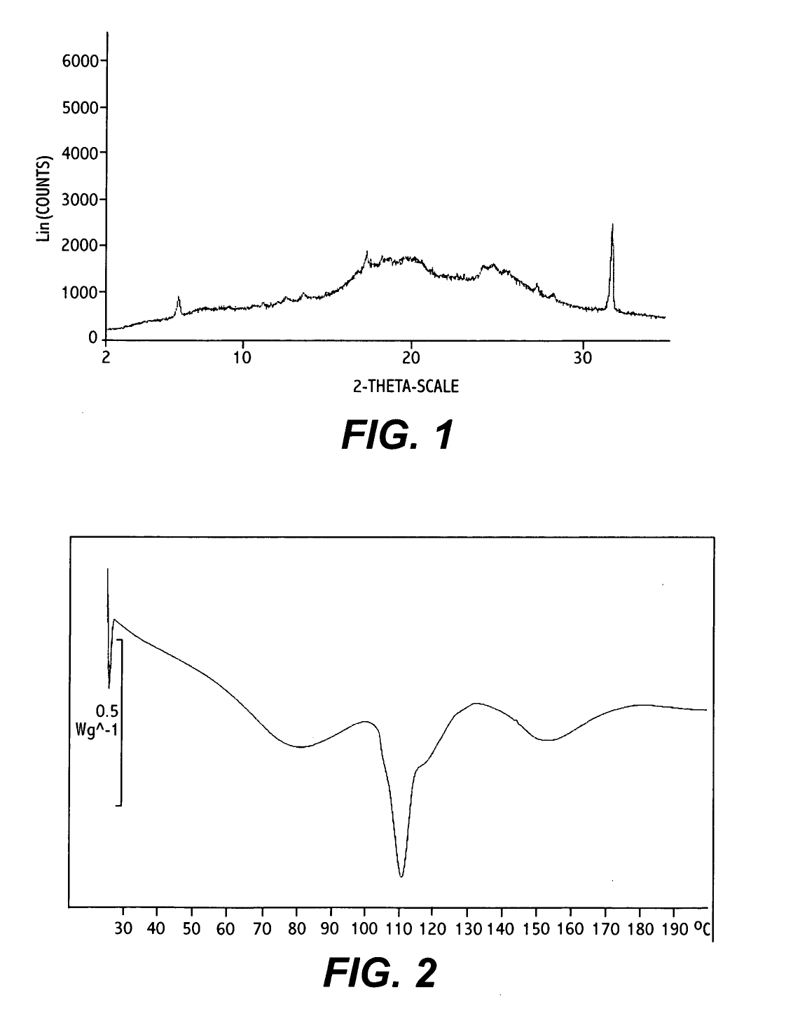

FIGS. 1 and 2 are the XRPD pattern and DSC curve, respectively, obtained for the yellow solid.

The filtrate was concentrated and re-dissolved in 50 ml of acetonitrile. After stirring at 4° C. overnight, a solid was collected by filtration and washing with acetonitrile (2×5 ml). 1.65 g of a solid was obtained after drying (purity 99.6% based on HPLC).

### Example 4

### Preparation of Crystalline Bosentan HCl Salt

A mixture containing 10.6 g of bosentan monohydrate in 50 ml of acetonitrile was warmed up to 50° C. With stirring, heating was stopped, 4 ml of an HCl solution (5 N to about 6 N, in isopropanol) was added dropwise and completed in about 2 min. The mixture was seeded and further stirred at room temperature for 2.5 hours. A solid was filtered out, washed with 5 ml acetonitrile, and dried in open air overnight. 8.85 g of a yellowish solid was obtained, with m.p. of about 150° C. to about 152° C. The crystalline structure was indicated by XRPD.

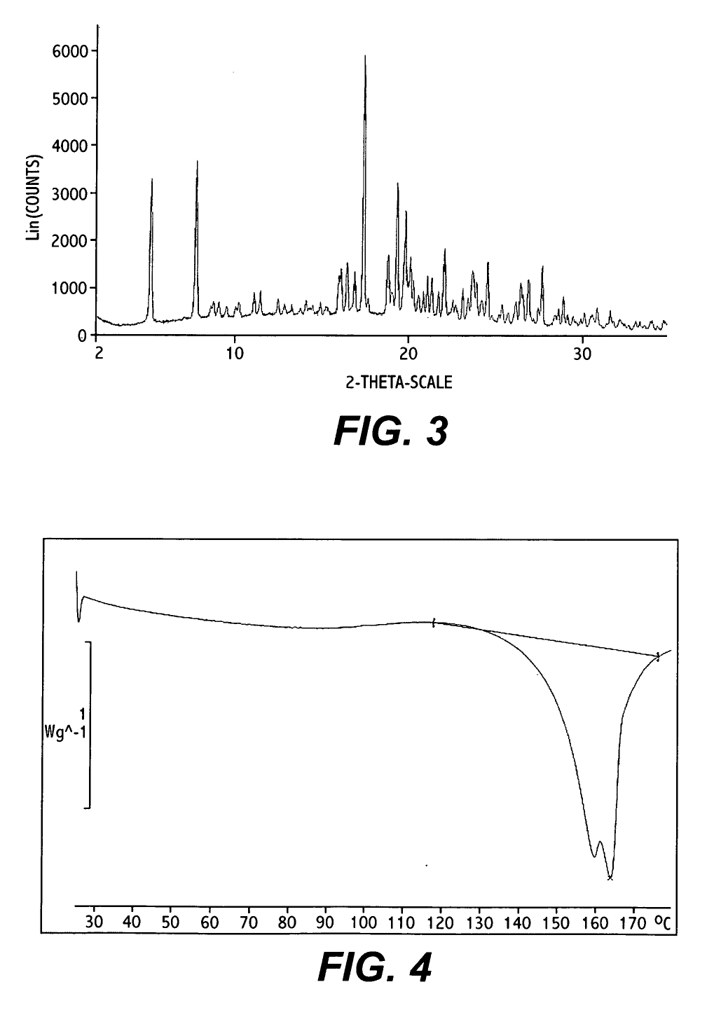

FIGS. 3 and 4 are the XRPD pattern and DSC curve, respectively, obtained for the yellowish solid.

### Example 5

### Preparation of Bosentan Monohydrate

12.5 g of the bosentan monohydrochloride salt (purity of about 99.0%) was suspended in 30 ml of ethanol. The mixture was warmed up to about 50° C. to obtain a clear solution. 20 ml of H₂O was added. The heating was stopped, and the mixture was neutralized to a pH of about 7 by the addition of a saturated NaHCO₃ solution. The formed suspension was further stirred for 2 hours. A solid was collected by filtration and washing (10 ml, ethanol/water=1/1). 10.95 g of a solid was obtained after drying at 40° C. in vacuo overnight, which showed a similar purity as the starting salt (about 99.0%).

### Example 6

### Preparation of Bosentan Hydrogensulphate

590 mg (about 1 mmole) of bosentan monohydrate (purity of about 99.46%, individual impurity of 0.38%) was dissolved in 10 ml of acetonitrile. At room temperature, while stirring, 100 mg (about 1 mmole) of sulphuric acid was added dropwise. The mixture was stirred for 3 hours at room temperature and further stirred at about 4° C. overnight.

A solid was filtered out and washed with 1 ml of acetonitrile. 550 mg of a yellowish solid was obtained after drying in open air overnight, which showed a purity of 99.84% and individual impurity of 0.16%.

M.p. about 165° C. to about 177° C.

### Example 7

### Preparation of Bosentan P-Toluenesulfonate

590 mg (about 1 mmole) of bosentan monohydrate (purity of about 99.46%, individual impurity of 0.38%) was dissolved in 10 ml acetonitrile. At room temperature, while stirring, 190 mg (about 1 mmole) of toluenesulphonic acid was added. The mixture was further stirred for 1.5 hours at room temperature.

A solid was filtered out and washed with 2 ml acetonitrile. 685 mg of a white solid was obtained after drying in open air overnight, which showed a purity of 99.78% and individual impurity of 0.22%.

M.p. about 205° C. to about 207° C.

### Example 8

### Preparation of Bosentan Oxalate

590 mg (about 1 mmole) of bosentan monohydrate (purity of about 99.46%, individual impurity of 0.38%) was dissolved in 10 ml of acetonitrile. At room temperature, while stirring, 90 mg (about 1 mmole) of oxalic acid was added. The mixture was further stirred for 1.5 hours at room temperature.

A solid was filtered out and washed with 1 ml of acetonitrile. 470 mg of a yellow solid was obtained after drying in open air overnight, which showed a purity of 99.62% and individual impurity of 0.24%.

M.p. about 173° C. to about 175° C.

### Example 9

### Preparation of Bosentan Maleate

590 mg (about 1 mmole) of bosentan monohydrate (purity of about 99.46%, individual impurity of 0.38%) was dissolved in 10 ml of acetonitrile. At room temperature, while stirring, 120 mg (about 1 mmole) of maleic acid was added. The mixture was stirred for 1.5 hours at room temperature and further stirred at about 4° C. overnight.

The solution was concentrated in vacuo to give a sticky oily material. 10 ml of ether was added and the mixture was triturated for 3 hours.

A solid was filtered out and washed with 2 ml of ether. 655 mg of a yellow solid was obtained after drying in open air overnight, which showed a purity of 99.44% and individual impurity of 0.32%.

M.p. about 154° C. to about 156° C.

### Example 10

### Preparation of Bosentan Hydrochloride

2.12 g of bosentan monohydrate was dissolved in 6 ml of isopropanol at reflux and 0.81 ml of 5 M HCl in isopropanol was added in one minute. The mixture was seeded with bosentan hydrochloride seeds and stirred overnight at room temperature. The solid was filtered and washed with ether. Dried at 40 C for 4 hours.

Yield: 2.01 g

### Example 11

### Preparation of Bosentan Methane Sulfonate

1600 mg of bosentan monohydrate was dissolved in 4 ml of isopropanol under reflux. 0.62 ml of methane sulfonic acid was added in one minute. The thick suspension was diluted with 10 ml of isopropanol. The mixture was stirred overnight, filtered, washed with ether and dried. M.p. 192 C (DSC)

### Example 12

### Preparation of Bosentan Benzene Sulfonate

1.6 g of bosentan monohydrochloride was dissolved in 4 ml of refluxing isopropanol, 0.501 g of benzenesulfonic acid was dissolved in refluxing isopropanol and added in one minute. The mixture was stirred at room temperature overnight, filtered and washed with ether and dried.

Each of the patents/applications mentioned above are incorporated herein by reference. The invention having been described it will be obvious that the same may be varied in many ways and all such modifications are contemplated as being within the scope of the invention as defined by the following claims.

We claim:

1. An acid addition salt of bosentan, wherein said salt is in solid state and wherein said acid has a pKa lower than 3.

2. The bosentan acid addition salt according to claim 1, wherein said salt is a monovalent salt having an acid:base ratio of about 1:1.

3. The bosentan acid addition salt according to claim 1, in crystalline form.

4. The bosentan acid addition salt according to claim 1, in amorphous form.

5. The bosentan acid addition salt according to claim 1, wherein said acid is selected from hydrochloric acid, hydrobromic acid, methane sulfonic acid, sulfuric acid, benzene sulfonic acid, p-toluenesulfonic acid, oxalic acid, and maleic acid.

6. The bosentan acid addition salt according to claim 1, wherein said salt is selected from bosentan hydrogensulphate, bosentan oxalate, bosentan maleate, bosentan hydrochloride, bosentan methane sulfonate, bosentan benzene sulfonate, and bosentan p-toluenesulfonate.

7. The bosentan acid addition salt according to claim 6, wherein said salt is selected from crystalline bosentan monohydrochloride and crystalline bosentan p-toluenesulfonate.

8. A pharmaceutical composition, comprising the bosentan acid addition salt of claim 1 and at least one pharmaceutically acceptable excipient.

9. A method of making an acid addition salt of bosentan, which comprises:
combining bosentan base and an acid having a pKa lower than 3 in an organic solvent to form a solution;
precipitating a bosentan acid addition salt from said solution; and
optionally isolating the precipitated bosentan acid addition salt.

10. The method according to claim 9, wherein said acid is selected from hydrochloric acid, hydrobromic acid, methane sulfonic acid, benzene sulfonic acid, sulfuric acid, p-toluenesulfonic acid, oxalic acid, and maleic acid.

11. The method according to claim 10, wherein said organic solvent is selected from the group consisting of C3-C10 aliphatic ketones; C1-C6 chlorinated hydrocarbons; C1-C6 aliphatic alcohols; C3-C10 aliphatic esters; C2-C5 aliphatic nitriles; ethers; and mixtures thereof.

12. The method according to claim 11, wherein said organic solvent is selected from the group consisting of acetone, methyl tert.butyl ketone, dichloromethane, methanol, ethanol, isopropanol, ethyl acetate, acetonitrile, di-isopropyl ether, tetrahydrofuran, and mixtures thereof.

13. A method of purifying bosentan, which comprises:
combining crude bosentan and an acid having a pK of about 3 or less in a first solvent to obtain an acid addition salt of bosentan;
isolating said acid addition salt of bosentan from said first solvent;
converting said bosentan acid addition salt into bosentan base in a second solvent; and
isolating said bosentan base from said second solvent.

14. The method according to claim 13, wherein said acid is selected from hydrochloric acid, hydrobromic acid, methane sulfonic acid, benzene sulfonic acid, sulfuric acid, p-toluenesulfonic acid, oxalic acid, and maleic acid.

15. The method according to claim 14, wherein said first solvent is selected from the group consisting of C3-C10 aliphatic ketones; C1-C6 chlorinated hydrocarbons; C1-C6 aliphatic alcohols; C3-C10 aliphatic esters; C2-C5 aliphatic nitrites; ethers; and mixtures thereof, and wherein said second solvent comprises water.

16. The method according to claim 15, wherein said second solvent is water.

17. The method according to claim 15, wherein said converting step comprises contacting said bosentan acid addition salt with an organic or inorganic base in said second solvent.

18. The method according to claim 15, which further comprises recrystallizing said isolated acid addition salt of bosentan prior to said converting step.

19. A process which comprises dissolving a solid bosentan acid addition salt according to claim 1 in an organic solvent, and precipitating said salt to obtain a purified solid bosentan acid addition salt.
